(19) United States
(12) Patent Application Publication
(10) Pub. No.: US 2006/0115443 A1
(43) Pub. Date: Jun. 1, 2006

(54) Cosmetic composition of two polysaccharides based on fucose and rhamnose

(76) Inventors: Jean-Luc Gesztesi, Sao Paulo (BR); Luciana Villa Nova Silva, Sao Paulo (BR); Ladislas Robert, Santeny (FR); Alexandre Michel Robert, Santeny (FR)

Correspondence Address: DORSEY & WHITNEY LLP;INTELLECTUAL PROPERTY DEPARTMENT, 250 PARK AVENUE, NEW YORK, NY, 10177, US

(21) Appl. No.: 10/552,375

(22) PCT Filed: Apr. 7, 2004
(86) PCT No.: PCT/FR04/00864

§ 371 Date: Dec. 6, 2005

(30) Foreign Application Priority Data
Apr. 8, 2003 (FR) 03/04336

(51) Int. Cl. A61K 8/73

(52) U.S. Cl. 424/70.13

(57) ABSTRACT

The invention relates to a cosmetic composition of two polysaccharides based on fucose and rhamnose, and to the use of the same especially in topically applied products which are used to act on cutaneous, epithelial and conjunctive tissue, especially for cosmetic anti-ageing effects.

The present invention relates to a novel cosmetic composition of two polysaccharides based on fucose and rhamnose and to its use especially in products for topical application, for which activity on the epithelial and connective skin tissue is desired, in particular cosmetic products with an antiaging effect.

In the course of aging, the skin becomes thinner by about 7% on average every ten years (Skin thickness changes in normal aging skin, Branchet et al., Gerontology, 1990, 36: 28-35). This thinning of the skin concerns the epidermis and the dermis. This loss of tissue represents an approximately equivalent loss of cells, keratinocytes from the epidermis and fibroblasts from the dermis. The keratinocytes present in the epidermis and the fibroblasts present in the dermis are known to produce proteolytic enzymes that contribute toward substantial thinning of the skin (Dr L. Robert: Le vieillissement [Aging], CNRS, Belin, 1994; Dr L. Robert: Le vieillissement, faits et theories [Aging, facts and theories], Dominos, Flammarion, 1995).

However, different macromolecules of the extracellular matrix (ECM) behave differently. The loss of collagen fibers has been shown as being slower than the loss of total skin tissue. Conversely, the biosynthesis of fibronectin has been shown as increasing in the skin with age and by fibroblasts in culture as a function of the number of passages. The elastic fibers of the papillary dermis, forming a vertical network, are gradually degraded. A majority of the horizontal elastic fiber networks predominate with age in the dermis. The surface density of the elastic fibers, quantified by image analysis, has been shown as increasing with age. This marked increase in the surface density of elastin with age is, however, accompanied by a gradual decline in the elasticity of the skin. These data show the complexity of the modifications dependent on the age, the skin tissue and its macromolecular composition.

One of the parameters mentioned above concerns the thickness of the skin, which decreases with age. The two layers of dermis and of epidermis are affected by this change.

In recent years, the object of numerous research studies has been to obtain compounds that are active against certain effects of aging of the skin. A first objective is to obtain thickening of the skin. Another objective is to slow down this process of thinning of the skin and to restore the normal thickness of the dermis.

Saccharides and polysaccharides are substances that are well known in cosmetics, especially for their moisturizing properties.

RROPs (rhamnose-rich oligo- and polysaccharides) are used as an aqueous solution containing 2.5% (w/w) of high molecular weight polysaccharides composed of 50% rhamnose. RROPs act on the adhesion to keratinocytes on the rhamnose-containing lectins and on inhibition of adhesion of the polynuclear leukocytes to the keratinocytes. RROPs thus make it possible to modulate the propagation of the cellular messages and consequently to attenuate the consecutive irritant reactions (anti-inflammatory effect).

FROPs (fucose-rich oligo- and polysaccharides) are oligo- and polysaccharides composed of polymers of a trisaccharide containing galactose, acetylgalacturonic acid and fucose, which acts on the fibroblasts of the dermis by stimulating their proliferation, by protecting them against the cytotoxicity induced by the free radicals emitted by ascorbate in the presence of Fe and EDTA (patent applications FR 2813 885 and FR 2 813 789).

It has now been found, surprisingly and unexpectedly, that a novel mixture of a rhamnose compound and a fucose compound has significant activities on various skin components, reflected by a clearly visible genuine antiaging result. This antiaging effect of the composition is attributed to the complementary properties of the RROPs acting on the keratinocytes at the skin surface and of the FROPs acting on the fibroblasts of the dermis, especially by inhibiting the free-radical degradation of hyaluronic acid, which plays in the dermis a fundamental role of filling and cohesion of the skin. The antiaging effect of the composition is also reflected by an increase in the thickness of the dermis and the epidermis.

### DESCRIPTION

Thus, one subject of the present invention is an antiaging cosmetic composition for the skin, characterized in that it comprises at least one rhamnose compound RROP, one fucose compound FROP and a cosmetically acceptable excipient.

The cosmetic composition according to the present invention may have variable percentages of fucose compound FROPs and of rhamnose compound RROPs, with, in general, a majority of fucose compound FROPs, which may range up to a composition containing equal parts of fucose compound FROPs and of rhamnose compound RROPs.

Preferably, the polysaccharide mixture according to the invention comprises 1/10 of rhamnose compound and 9/10 of fucose compound.

More particularly, the polysaccharide mixture according to the invention comprises ⅕ of rhamnose compound and ⅘ of fucose compound.

More particularly, the polysaccharide mixture according to the invention comprises ⅓ of rhamnose compound and ⅔ of fucose compound.

Even more particularly, the polysaccharide mixture according to the invention comprises ½ of rhamnose compound and ½ of fucose compound.

The polysaccharide mixture is most particularly suitable as active agent in a cosmetic composition, especially an antiaging composition (topical application). In particular, the observed biological effects of this polysaccharide mixture prove to be comparable with or even greater than those of the polysaccharides taken separately, their actions being found to be complementary and synergistic.

The cosmetically acceptable excipient may be any excipient among those known to a person skilled in the art for the purpose of obtaining a composition according to the invention in the form of a cream, a lotion, a gel, a pomade, etc., optionally in the form of an emulsion also with components known to those skilled in the art, to improve, modify or stabilize the composition from a cosmetic viewpoint.

By way of example, the composition according to the invention may comprise excipients that are well known to those skilled in the art for the formulation of a composition intended for topical application. Such excipients may be chosen from the group consisting of skin-structuring agents (such as squalene and sphingolipids), humectants (such as glycerol and hydroxyprosilane C), emollients (such as butylene glycol and cetyl lactate), silicones (such as cyclomethicone), antisun agents (such as Parsol 1789 and Eusolex 6300), emulsifiers (especially Carbopol 1342 combined with triethanolamine and soybean lecithin), thickeners (especially xanthan gum), sequestering agents (especially EDTA), antioxidants (such as BHT), fragrances, preserving agents and water, and mixtures thereof.

The oligosaccharide mixture is used in a proportion of between about 0.1% and 10% by weight relative to the total weight of the cosmetic composition.

Needless to say, the operating conditions for preparing the cosmetic composition according to the invention form part of the general knowledge of a person skilled in the art.

### KEY TO THE FIGURES

FIG. 1 is a curve showing the results of the efficacy of free-radical uptake by the FROPs and RROPs presented in example 1, in terms of a percentage of inhibition of free-radical degradation of hyaluronic acid on skin explants by concentration of FROPs and RROPs.

FIG. 2 is a curve showing the results for the degradation of hyaluronic acid in the presence of free radicals and/or of RROPs presented in example 2, using a viscosimetric measurement based on the release of free radicals (°H) by ascorbate in the presence of Fe and EDTA.

### EXAMPLE 1

In this example, the capacity for uptake of free radicals by the RROPs and FROPs was compared using a viscosimetric measurement based on the release of free radicals (°OH) by ascorbate in the presence of Fe and EDTA.

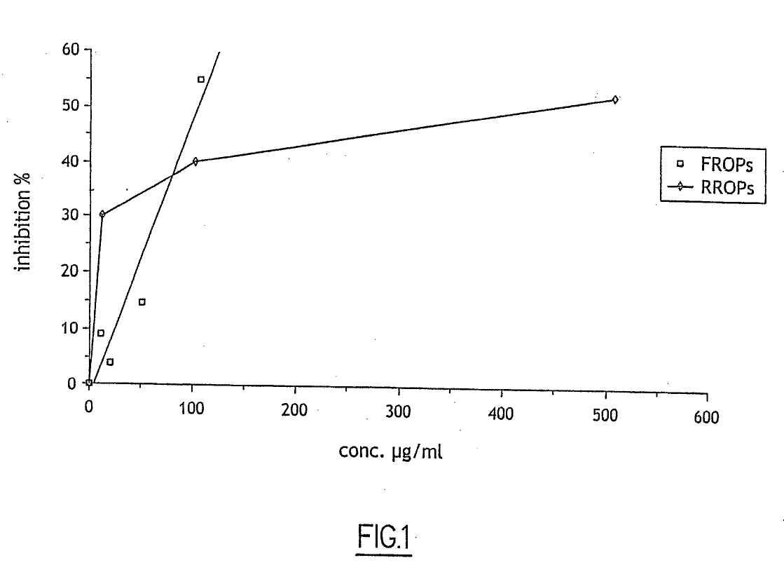

FIG. 1

FIG. 1 shows the dose-dependent inhibition by FROPs and RROPs of the free-radical degradation of hyaluronic acid.

It is observed that the RROPs show substantial inhibition at a low concentration (less than 10 μg/ml), but that this effect reaches a plateau at higher concentrations, after 100 μg/ml.

It is observed that the FROPs show low inhibition at low concentrations (less than 20% inhibition at up to 50 μg/ml). However, the inhibition increases linearly at higher concentrations and exceeds the inhibition observed for the RROPs: at 100 μg/ml the inhibition is 38% for the RROPs and 55% for the FROPs. In conclusion, these results suggest that at concentrations relative to the composition of the mixture of FROPs and RROPs of the present invention, there is a synergistic effect of these two polysaccharides on the uptake of free radicals.

### EXAMPLE 2

In this example, the variation in the viscosity of hyaluronic acid in the presence of free radicals and/or of RROPs was measured using a viscosimetric measurement based on the release of free radicals (°OH) by ascorbate in the presence of Fe and EDTA.

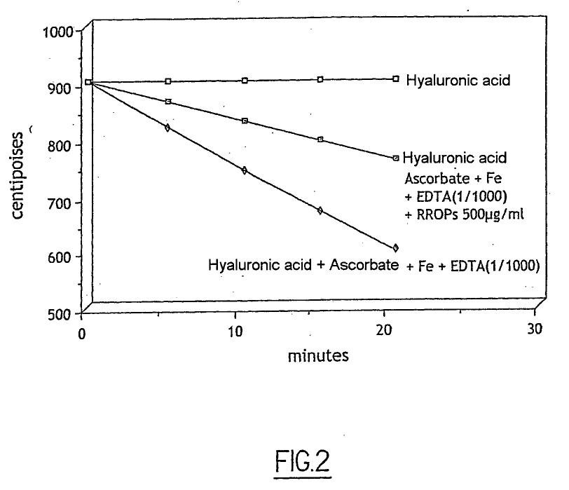

FIG. 2

FIG. 2 shows the degradation of hyaluronic acid by the free radicals released by ascorbate in the presence of Fe and EDTA as a function of exposure time: 1) in the absence of free-radical-releasing agent, 2) in the presence of such agents at 1/1000, and 3) in the presence of such agents at 1/1000 and of RROPs at 500 μg/ml.

It is observed that the variation in viscosity between the hyaluronic acid alone and in the presence of free radicals is 14.5 cpoises/minute, whereas it is only 7.0 cpoises/minute in the presence of RROPs. The percentage of inhibition by the RROPs of the degradation of hyaluronic acid by the free-radical-releasing agent is thus 52%.

It is concluded that RROPs have a protective action on hyaluronic acid with respect to free radicals.

### EXAMPLE 3

In this example, we examined the effects of a local application of RROPs (Rhamnosoft®) and of FROPs (Elastinol®) and the mixture of the two on the thickness of the skin of hairless rats.

Materials and Methods

Animals and Treatments

10 female hairless rats with an average initial weight of 170 g were used for this study. These 10 animals are divided into 3 groups:

the first group includes 3 rats (Nos. 1, 2 and 3)

the second group also includes 3 rats (Nos. 4, 5 and 6), and finally

the third group comprises 4 rats (Nos. 7, 8, 9 and 10).

The rats are kept in individual cages and have free access to water and industrial rat feed. The left side of these animals is used for control and treated only with the base preparation used as vehicle: BioDerma “Biobase Creme H/E”. The right side of the animals is treated with the same base also containing the following active principles: 0.25% Rhamnosoft for the first group, 0.75% Elastinol for the second group and a mixture of the two polysaccharides in the same proportions, giving a final concentration of 1% for the third group. 1 g of these three preparations is administered locally, 5 days a week for 4 weeks. The penetration of these preparations is performed by spreading the product and rubbing it in for approximately one minute on each side.

Collection of the Skin Samples

After the treatment period, the animals are sacrificed by means of injection of a lethal dose of pentobarbital anesthetic. In order to avoid contraction of the skin after collection, plastic rings 10 mm in diameter are bonded to the skin of the rats using cyanoacrylate glue (cyanolithe). The skin is then cut around the ring (0.785 cm²) and removed. A second ring is bonded onto the inner surface of the skin, exactly opposite the other ring. These circular skin samples are then fixed in a Bouin solution for 24 hours, followed by washing, dehydration, impregnation with paraffin solvents, and they are then included into paraffin containing a synthetic polymer (paraplast). From each block of paraplast, 12 slices 5 μm thick are made using a Reichert microtome. Two of these slices are stained with hematoxylin and eosin (HE), two histological stains. Such slices stained with HE are used to measure the thickness of the dermis.

Evaluation of the Thickness of the Dermis

This evaluation is performed on the slices stained with hematoxylin/eosin, observed on a Zeiss photomicroscope equipped with a black and white video camera, connected to a Nixdorf Power Tower microcomputer containing the Visiolab 1000 image analysis software from Biocom (France). The semiautomatic length measuring function is used, and the results are expressed in pixels (image points). The magnification ratio is 20×. The calibration is performed by measuring known lengths on a Malassez cell, which allows the conversion of the values in pixels into microns. To obtain the thickness of the dermis, perpendicular lines are plotted from the dermo-epidermal basal lamina to the upper limit of the hypodermis. 10 microscopic fields selected at random were analyzed for each skin sample, with 25 measurements per field, which gives a total of 250 measurements for each sample. The individual measurements and the means±standard deviation for each field are recorded and used for the final evaluation of the results.

As all 10 rats have a control side, the control values are the means of 2500 individual measurements. As the first two groups are composed of only 3 rats, the means for these groups are obtained on 750 measurements. Finally, for the group treated with the mixture of the two polysaccharides, the mean is based on 1000 individual measurements. Such a high number of measurements gives a high degree of safety to the statistical evaluation of the results, performed using the StatView software and the Student t test.

Result of the Thickness of the Dermis

For the dermis thickness measurements, a 2.5× objective lens is used, giving a final magnification of 20×. Table 2 shows the results obtained, expressed as pixels (image points).

TABLE 1

| | Mean | Standard | Student t test |
|---|---|---|---|
| Treatment of | value in | error of | compared with |
| the rats | pixels | mean | the controls |
| Controls | 172.21 | 4.67 | |
| Rhamnosoft | 182.96 | 5.52 | N.S. |
| Elastinol | 179.96 | 5.63 | N.S. |
| Elastinol/Rhamnosoft | 199.19 | 2.88 | p < 0.0001 |
| mixture | | | |

It is seen that the three means corresponding to the dermides of the treated skin samples are more or less higher than the control value. The mean thickness of the control aermides is 172.21±4.67 pixels.

For the dermides treated with Rhamnosoft, the corresponding figure is 182.96±5.52 pixels. This corresponds to an increase in the thickness of the dermis of 6.2% relative to the control value, but this difference is not statistically significant.

For the dermides treated with Elastinol, the mean thickness obtained is 179.96±5.63 pixels, which is only 4.5% higher than the control value. This difference is not statistically significant either.

The mean thickness of the dermis after 4 weeks of treatment with a mixture of Rhamnosoft and Elastinol is 199.19±2.88 pixels. This increase in the thickness of the dermis treated with this mixture, compared with the control value, is statistically highly significant: p<0.0001.

Discussion

The results presented above indicate that, at low concentrations, neither Rhamnosoft (0.25%) nor Elastinol (0.75%) alone significantly increases the thickness of the dermis of the skin of the treated rats. Conversely, when the mixture of both of these polysaccharides is used for the treatment, each being present at the concentration used individually, a statistically significant increase in the thickness of the dermis is observed, corresponding to roughly 16% of the thickness of the control dermis. This increase is greater than the sum of the individual effects of the two polysaccharides tested (6.2±4.5=10.7) by virtually 50%, and this suggests that the two test substances potentiate their actions, or more simply that they have a synergistic effect on the thickness of the dermis.

### EXAMPLE 4

We have shown in the preceding example that the combination of FROPs and RROPs allows a statistically significantly large increase in the thickness of the dermis. In the present example, we have compared by semiautomatic morphometry the effect on the cell density of the epidermis of FROPs (Elastinol®) and RROPs (Rhamnosoft®) or the mixture of the two.

Materials and Methods

Animal Experimentation

Animals and Treatments

10 female hairless rats with an initial weight of 170 g were used. They were kept in individual cages, with free access to water and industrial rat feed. The left side of the animals was used as control and treated only with the base preparation used as vehicle. The right side was treated differently in the three groups of rats.

The first group, rats Nos. 1, 2 and 3: 0.25% Rhamnosoft The second group, rats Nos. 4, 5 and 6: 0.75% Elastinol The third group, rats Nos. 7, 8, 9 and 10: 0.25% Rhamnosoft+0.75% Elastinol

1 g of these preparations was administered locally, 5 days a week for 4 weeks. The penetration of these preparations was ensured by spreading and rubbing them in for approximately one minute on each side.

Collection of the Skin Samples

After the administration period, the animals are sacrificed by means of injection of a lethal dose of pentobarbital anesthetic. In order to avoid contraction of the skin after collection, plastic rings 10 mm in diameter are bonded to the skin of the rats using cyanoacrylate glue (cyanolithe). The skin is then cut around the ring (0.785 cm²) and removed. A second ring is bonded onto the inner surface of the skin, exactly opposite the other ring. These circular skin samples are then fixed in a Bouin solution for 24 hours, followed by washing, dehydration, impregnation with paraffin solvents, and they are then included into paraffin containing a synthetic polymer (paraplast) From each block of paraplast, 12 slices of 5 μm were prepared with a Reichert microtome, and mounted onto slides. Two of them were stained with hematoxylin/eosin (HE), two histological stains. Such HE-stained slices were used for the measurement of the cell density of the epidermis.

Evaluation of the Cell Density

This evaluation is performed on the slices stained with hematoxylin/eosin, observed on a Zeiss photomicroscope equipped with a black and white video camera, connected to a Nixdorf Power Tower microcomputer containing the Visiolab 1000 image analysis software from Biocom (France). The manual contour extraction function is used, and the results are expressed in pixels (image points). The magnification ratio is 320×. Firstly, we measured the explored surface area, expressed in pixels. Next, we manually extracted all the perimeters of all the cells present in the microscopic field. The software calculated from these measurements for each field the number of cells, the surface area of each cell and the number of pixels containing a cell, and also the means of these values for each sample studied. 10 microscopic fields selected at random were analyzed for each skin sample. The statistical evaluation of the results was performed with the StatView software and the Student t test.

Results

The results obtained, expressed in pixels, are given in tables 3, 4 and 5, one per administered treatment. 4 parameters are indicated in these tables. The first is the explored surface area (in pixels±the standard error of mean). The second gives the number of cells observed on the explored surface area. The third parameter is the surface area, always expressed in pixels, which contains a cell, thus giving the possibility of numerically expressing the cell density of the studied sample. Finally, the variation of the mean surface area of the cells can indicate a possible trophic effect on the cells.

TABLE 2
Results obtained on rats treated
with Rhamnosoft (Nos. 1, 2 and 3)

| | Explored | | | |
|---|---|---|---|---|
| | surface | No. of | Pixels per | Mean cell |
| | areas | cells | cell | surface area |
| Controls | 50 909 ± | 31.7 ± 1.27 | 1648.07± | 415.6 ± |
| | 1820 | | | 11.65 |
| Rhamnosoft | 64 279 ± | 42.36 ± 2.97 | 1529 ± 65.23 | 494.93 ± |
| | 4609 | | | 20.38 |
| t Test | p < 0.002 | p < 0.000 | p < 0.1 + 61 | p < 0.001 |
| | | | N.S. | |

It is seen from the table that, in the skin slices treated with Rhamnosoft, the number of cells is significantly higher than the corresponding number in the controls. This is likewise the case for the mean surface area of the cells. On the other hand, the number of pixels containing a cell does not differ significantly between treated and control. This means that the cellularity of the epidermis treated with Rhamnosoft is of the same order as that of the control epidermis. However, a significant trophic effect on the cells in the treated skin is seen.

TABLE 3
Results expressed in pixels obtained for the
rats treated with Elastinol (Nos. 4, 5 and 6)

| | Explored | | | Mean cell |
|---|---|---|---|---|
| | surface | No. of | Pixels per | surface |
| | area | cells | cell | area |
| Controls | 50 909 ± | 31.7 ± 1.27 | 1648.07± | 415.6 ± 11.65 |
| | 1820 | | | |
| Elastinol | 56 987 ± | 36.00 ± 2.78 | 1556.02 ± | 468.53 ± 31.8 |
| | 6931 | | 79.61 | |
| t Test | p < 0.227 | p < 0.145 | p < 0.360 | p < 0.000 |
| | N.S. | N.S. | N.S. | |

The measurement results shown in table 4 do not show any statistically significant differences between the controls and the samples treated with Elastinol, either as regards the explored surface area, or as regards the number of cells counted on the explored surface area, or as regards the number of pixels containing a cell. Only the difference in the mean surface area of the cells is significant: treated samples: 468.53, versus control: 415.60; p<0.000.

TABLE 4
Results expressed in pixels obtained for the rats treated
with Elastinol + Rhamnosoft (Nos. 7, 8, 9 and 10)

| | Explored | | | Mean cell |
|---|---|---|---|---|
| | surface | No. of | Pixels per | surface |
| | area | cells | cell | area |
| Controls | 50 909 ± | 31.7 ± 1.27 | 1648.07 ± | 415.6 ± 11.65 |
| | 1820 | | 45.6 | |
| Elast + rhamno | 53 853 ± | 34.44 ± 1.51 | 1623.80 ± | 497.88 ± 23.53 |
| | 2612 | | 111.1 | |
| t Test | p < 0.387 | p < 0.236 | p < 0.810 | p < 0.001 |
| | N.S. | N.S. | N.S. | |

As may be seen in this table, the differences between the control and the rats treated with Elastinol+Rhamnosoft are not significant for the first three parameters. Only the mean surface area of a cell is significantly higher for the treated samples compared with the controls.

### DISCUSSION, CONCLUSION

Considering that only the results statistically different than the controls may be retained, it is observed that only the study of the mean surface area of the cells corresponds to this criterion, with the application of Rhamnosoft alone and the application of the Rhamnosoft+Elastinol combination. It is again the mixture of the two polysaccharides (RROPs and FROPs) that makes it possible to obtain the greatest increase in the mean surface area of the cells, and thus the greatest antiaging effect on the epidermis.

1. An antiaging cosmetic composition for the skin, characterized in that it comprises at least one rhamnose compound RROPs, one fucose compound FROPs and a cosmetically acceptable excipient.

2. The cosmetic composition as claimed in claim 1, characterized in that it comprises, relative to the total weight of the polysaccharide mixture, 1/10 of rhamnose compound RROPs and 9/10 of fucose compound FROPs.

3. The cosmetic composition as claimed in claim 1, characterized in that it comprises, relative to the total weight of the polysaccharide mixture, ⅕ of rhamnose compound RROPs and ⅘ of fucose compound FROPs.

4. The cosmetic composition as claimed in claim 1, characterized in that it comprises, relative to the total weight of the polysaccharide mixture, ⅓ of rhamnose compound RROPs and ⅔ of fucose compound FROPs.

5. The cosmetic composition as claimed in claim 1, characterized in that it comprises, relative to the total weight of the polysaccharide mixture, ½ of rhamnose compound RROPs and ½ of fucose compound FROPs.

6. The cosmetic composition as claimed in claim 1, characterized in that it is suitable for topical application, especially in that it is in the form of a cream.

7. The cosmetic composition as claimed in claim 1, characterized in that the oligosaccharide mixture is used in a proportion of between about 0. 1% and 10% by weight relative to the total weight of the composition.

8. The use of the cosmetic composition as claimed in claim 1, characterized in that it is intended for antiaging action on the skin.

9. A cosmetic treatment method for the skin, characterized in that a cosmetic composition comprising at least one polysaccharide mixture as defined in claim 1 is applied to the skin.
